(19) United States
(12) Patent Application Publication
(10) Pub. No.: US 2023/0200707 A1
(43) Pub. Date: Jun. 29, 2023

(54) VENTRICULAR DRAIN OR SHUNT ASSSEMBLY WITH RECORDING ELECTRODE

(71) Applicant: Cerebral Therapeutics, Inc., Aurora, CO (US)

(72) Inventors: Daniel J. Abrams, Denver, CO (US); Matias Maturana, Melbourne (AU); Ross Thomas, Victoria (AU); Christopher Smith, Victoria (AU)

(21) Appl. No.: 18/088,854

(22) Filed: Dec. 27, 2022

Related U.S. Application Data
(60) Provisional application No. 63/294,611, filed on Dec. 29, 2021.

(51) Int. Cl. A61B 5/293; A61M 27/00; A61B 5/00

(52) U.S. Cl. CPC A61B 5/293; A61B 5/6864; A61M 27/006; A61B 2562/0209; A61M 2027/004

(57) ABSTRACT

A device is configured to receive a catheter. The catheter may be a catheter of a ventriculoperitoneal shunt or an external ventricular drain. The device includes (ii) an upper flange having an opening, and (ii) elongate body defining a lumen in communication with the opening. The catheter may be inserted through the opening and the lumen. The device includes an electrode disposed on the elongate body. When the device is implanted in a subject, the upper flange rests on a skull of the subject adjacent to a burr hole, the elongate body is inserted through the burr hole, and the electrode is positioned intracranially in the subject.

### CROSS REFERENCE TO RELATED APPLICATION

This application claims the benefit of priority to U.S. Provisional Pat. Application No. 63/294,611, filed on 29 Dec. 2021, which application is hereby incorporated herein by reference in its entirety.

### FIELD

The present disclosure relates to, among other things, devices, assemblies, and systems having recording electrode for transmitting, collecting, analyzing, or monitoring intracranial electroencephalogram (iEEG) data; particularly for use with an externalized ventricular drain (EVD) or ventriculoperitoneal (VP) shunt, as well as methods associated therewith.

### INTRODUCTION

Each year, tens of thousands of EVDs and VP shunts (VPSs) are implanted in patients in the United States to reduce intracranial pressure (ICP). EVDs are typically placed to manage elevated ICP associated with traumatic brain injury (TBI). VPSs may be placed to manage elevated ICP with, for example, post-traumatic hydrocephalus or congenital hydrocephalus.

EVDs are explanted before the patient leaves the intensive care unit (ICU) or is discharged from the hospital, typically within a few days, weeks, or months of being implanted. VPSs are intended for longer term or permanent implantation. However, for various reasons, VPSs have about a 25% revision rate, resulting in a large number of revision surgeries, explanation, and replacement of the VPS with a new VPS.

The ability to monitor brain activity in patients in which EVDs and VPSs are implanted is limited. Existing methods for monitoring brain activity, such as magnetic resonance imaging (MRI), functional MRI (fMRI), electroencephalogram (EEG), and magnetoencephalogram (MEG) recordings, as well as other imaging techniques, are unable to continuously measure high quality temporally relevant brain data over long periods of time.

It would be desirable to continuously measure high quality temporally relevant brain data over long periods of time in patients in which EVDs and VPSs are, or have been, implanted. As EVDs and VP shunts may be explanted or revised, it would be desirable to continue monitoring brain activity and recording data after the devices are explanted or replaced.

### SUMMARY

The present disclosure relates to, among other things, devices, assemblies, and systems that permit implantation of an intracranial EEG (iEEG) recording electrode during a procedure in which an EVD or VPS is implanted. In some embodiments, the devices, assemblies, and systems are configured such that the iEEG recording electrode remains implanted after the EVD or VPS is explanted or replaced. In some embodiments, the devices, assemblies, and systems are configured to permit revision of a VPS or portion thereof without removing the iEEG recording electrode.

In some aspects, the present disclosure describes a device comprising (i) an upper flange comprising an opening configured to receive a catheter; (ii) an elongate body defining a lumen configured to receive the catheter, wherein the lumen is in communication with the opening of the upper flange; and an electrode disposed on the elongate body. When the device is implanted in a subject, the upper flange is configured to rest on a skull of the subject adjacent to a burr hole, the elongate body is configured to be inserted through the burr hole, and the electrode is configured to be positioned intracranially in the subject. The electrode is configured to detect intracranial electroencephalogram (iEEG) signals from the subject

The details of one or more aspects of the disclosure are set forth in the accompanying drawings and the description below. Other features, objects, and advantages of the techniques described in this disclosure will be apparent from the description and drawings, and from the claims.

### DEFINITIONS AND CONTEXT FOR SOME DEFINED TERMS

All scientific and technical terms used herein have meanings commonly used in the art unless otherwise specified. The definitions provided herein are to facilitate understanding of certain terms used frequently herein and are not meant to limit the scope of the present disclosure.

As used herein, “disposed on” in the context of an electrode on a body, such as an elongate body refers to the electrode being associated with the body in a manner such that the position of the electrode maintains a position relative to the body and at least a portion of the electrode is exposed to tissue or fluid of the subject when the body is implanted in the subject. The electrode may be bonded, fastened, or adhered to the body, may be recessed into a portion of the body, or otherwise retained by the body, or the like.

As used herein, “diameter” refers to the largest distance from one edge to another of an object along a section orthogonal to a longitudinal axis of the object. If the object is cylindrical, the diameter will be constant along the section. If the object is a rectangular prism having a square cross-section, the diameter is the distance from one corner of the square to the opposing corner of the square.

The terms “coupled” or “connected” refer to elements being attached to each other either directly (in direct contact with each other) or indirectly (having one or more elements between and attaching the two elements). Either term may be replaced to “couplable” or “connectable” to describe that the elements are configured to be coupled or connected. In addition, either term may be modified by “operatively” and “operably,” which may be used interchangeably, to describe that the coupling or connection is configured to allow the components to interact to carry out functionality.

As used herein, “treat,” “treatment,” or the like mean to reduce or alleviate one or more symptom or to slow the progression of the disease being treated.

As used herein, “intracranial” means within the skull of a subject. An intracranial electrode may be placed at any suitable location within the confines of a skull of a subject. For example, the intracranial electrode may be placed in the brain of the subject or on a surface of the brain of the subject. An intracranial electrode may be placed in brain parenchyma (“intraparenchymal”). An intracranial electrode may be placed within the cerebrum of a subject (“intracerebral”). An intracranial electrode may be placed within an intracranial blood vessel (“intravascular”). An intracranial electrode may be placed within an cerebral ventricle (“intraventricular”). iEEG signals include signals obtained from electrodes positioned on the surface of the brain of a subject and within the brain of the subject, including electrodes placed intraparenchymally, intracerebrally, intraventricular, and intravascularly.

As used herein, a “brain state” is a symptom or function of the brain that (i) involves multiple areas and neuronal networks of the brain and (ii) is reflected in brain activity. A brain state may be manifest in normal or abnormal brain activity. Motor function, speech function, visual function, somatosensory sensation function, smell function, and the like, in and of themselves, are not brain states. These functions, in and of themselves, impact smaller, more discrete, less distributed regions of the brain. For example, visual function involves the visual cortex, visual radiations, and optic tract. A brain state may, however, involve one or more of motor function, speech function, visual function, smell function, and the like. A brain state may involve activity in 2 or more Brodmann areas, 3 or more Brodmann areas, 4 or more Brodmann areas, 5 or more Brodmann areas, 6 or more Brodmann areas, 7 or more Brodmann areas, 8 or more Brodmann areas, 9 or more Brodmann areas, or 10 or more Brodmann areas.

As used herein, a “psychological brain state” is a brain state having a mental or emotional component. A subject is typically aware of their psychological brain state in their feelings and thoughts. Examples of psychological brain states include general affect or mood, anxiety, depression, addiction, obsession, suicidal thoughts, hallucinations, cognition, attention, post-traumatic stress, and the like, and degrees thereof. As an example, a traumatic memory may involve one area of the brain, but post-traumatic stress disorder (PTSD) is the impact of that memory which distracts attention, causes fear, and/or impacts cognitive function. Accordingly, PTSD involves or impacts multiple regions and neural networks of the brain.

Psychological brain states do not include seizure activity and/or motor activity alone. However, electrical brain activity associated with epileptic and/or motor activity may be relevant to a broader psychological brain state.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, singular forms “a,” “an” and “the” include plural referents unless the context clearly dictates otherwise.

As used herein, the term “or” is generally employed in its sense including “and/or” unless the content clearly dictates otherwise. The term “and/or” means one or all the listed elements or a combination of any two or more of the listed elements. The use of and/or″ in some locations of the present disclosure is not intended to mean that the use of “or” in other locations cannot be interpreted as “and/or.”

The phrases “at least one of” and “one or more of” followed by a list refers to any one of the items in the list and any combination of two or more items in the list.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

Any direction referred to herein, such as “top,” “bottom,” “side,” “upper,” “lower,” and other directions or orientations are described herein for clarity and brevity but are not intended to be limiting of an actual device or system. Devices and systems described herein may be used in a number of directions and orientations.

As used herein, “providing” an article, device, or system means manufacturing the article, device, or system, assembling the article, device, or system, purchasing the article, device, or system, or otherwise obtaining the article, device, or system.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must).

The words “include,” “including,” and “includes” indicate open-ended relationships and therefore mean including, but not limited to. Similarly, the words “have,” “having,” and “has” also indicated open-ended relationships, and thus mean having, but not limited to. Similarly, the terms “comprise” and “comprising” indicate open-ended relationships, and thus mean comprising, but not limited to. The terms “consisting essentially of” and “consisting of” are subsumed within the term “comprising.” For example, a catheter comprising tubing may be a catheter consisting of tubing. The term “consisting essentially of” means a recited list of one or more items belonging to an article, kit, system, or method and other non-listed items that do not materially affect the properties of the article, kit, system, or method.

The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a catheter connector may be configured to place a lumen of a catheter in fluid communication with a fluid path, even when the catheter is not connected to the catheter connector).

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

### BRIEF DESCRIPTION OF THE DRAWINGS

FIG. 1 is a schematic side view of an embodiment of a device shown relative to a skull.

FIG. 2 is a schematic top-down view of an embodiment of a device.

FIGS. 3A-C are schematic sectional views of embodiments of a device.

FIG. 4 is a schematic top-down view of an embodiment of a device.

FIGS. 5 and 6 are schematic side views of embodiments of a device shown relative to a skull.

FIGS. 7A, 8A, and 9A are schematic sectional views of embodiments of a device. Electrodes and conductors are not shown.

FIGS. 7B, 8B, and 9B are schematic perspective see-through views of embodiments of a device. Elongate bodies are not shown. Electrodes and conductors are not shown.

FIGS. 7C, 8C, and 9C are schematic perspective sectional views of embodiments of devices showing upper flange portions and mid-portions. Elongate bodies are not shown. Electrodes and conductors are not shown.

FIG. 10 is a schematic side view of a portion of an embodiment of a ventriculoperitoneal shunt (VPS).

FIG. 11 is a schematic side view of an embodiment of a VPS.

FIGS. 12 and 13 are schematic perspective views of embodiments of a device.

FIG. 14 is a schematic view of components of a system implanted in a subject.

FIG. 15 is a schematic view of components of a system shown relative to a skull.

FIG. 16 is a schematic view of a components of a system including an external device.

While the invention is susceptible to various modifications and alternative forms, specific embodiments thereof are shown by way of example in the drawings and may herein be described in detail. The schematic drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

### DETAILED DESCRIPTION

The present disclosure relates to, among other things, devices, assemblies, and systems including a catheter for insertion into a brain of a subject and a recording electrode for transmitting, collecting, analyzing, or monitoring intracranial electroencephalogram (iEEG) data, as well as methods associated therewith.

The systems described herein may include any suitable catheter. In some embodiments, the catheter is configured (e.g., has an appropriate length, is formed from suitable materials, has suitable properties regarding flexibility, etc., and the like) to be implanted in a cerebrospinal fluid (CSF) space of a subject. For example, the catheter may comprise a distal end configured to be inserted into a cerebral ventricle, such as a lateral ventricle. The catheter may be a catheter of an external ventricular drain (EVD) or ventriculoperitoneal shunt (VPS).

In embodiments, a device comprises an electrode configured to detect the electroencephalogram (EEG) signals. The device is configured to receive therethrough the catheter. The device is configured such that, when implanted, the electrode is intracranially positioned. Preferably, the device is configured such that the catheter may be explanted or withdrawn from the device while maintaining the intracranial position of the electrode. In embodiments, a second catheter may be inserted through the device after the first catheter is withdrawn.

The device may comprise an upper flange, an elongate body, and an electrode disposed on the elongate body. The upper flange may comprise an opening configured to receive a catheter. The elongate body may comprise a lumen configured to receive the catheter. The lumen may be in communication with the opening in the upper flange. The catheter may be inserted through the device by inserting the catheter into the opening and through the lumen. The upper flange is configured to rest on a skull of the subject adjacent a burr hole when the device is implanted. The upper flange is configured to be implanted between the skull and a scalp of the subject. The elongate body may be inserted through the burr hole such that the electrode is positioned intracranially.

Preferably, the opening of the upper flange and the lumen of the elongate body define an inner diameter greater than a diameter of the catheter to allow the catheter to be easily inserted and removed from the device. The opening and the lumen may define an inner diameter 0.1 mm or more greater than an outer diameter of the catheter, such as 0.2 mm or more, 0.3 mm or more, 0.4 mm or more, 0.5 mm or more, 0.6 mm or more, 0.7 mm or more, 0.8 mm or more, 0.9 mm or more, or 1 mm or more greater than the diameter of the catheter.

In embodiments, the diameter of the opening in the upper flange is greater than the diameter of lumen of the elongate body. For example, the diameter of the opening may be 2 mm or more or 3 mm or more greater than the diameter of lumen of the elongate body.

The diameter of the lumen of the elongate body is preferably no more than 1 mm greater than the outer diameter of the catheter to keep minimal the outer diameter of the elongate body, particularly if the elongate body is configured to penetrate the brain of the subject when implanted. In embodiments, the diameter of the lumen is no more than 0.9 mm, no more than 0.8 mm, no more than 0.7 mm, no more than 0.6 mm greater, no more than 0.5 mm greater, no more than 0.4 mm greater, no more than 0.3 mm greater, no more than 0.2 mm greater, or no more than 0.1 mm greater than the outer diameter of the catheter. The diameter of the lumen may be substantially the same (e.g., within 10 %) along the length of the elongate body or may vary along the length of the elongate body.

In embodiments, the diameter of the lumen of the elongate body is from about 2 mm to about 5 mm, such as from about 2.2 mm to about 4 mm, or from about 2.5 mm to about 3.5 mm.

The material of the elongate body defining the lumen and the material defining an outer surface of the catheter preferably have a low coefficient of friction to allow the catheter to easily slide through the lumen. Preferably, the lumen of the elongate body comprises a smooth surface. The surface of the elongate body that defines the lumen may be lubricious or a lubricious coating may be applied to the surface of the lumen.

The elongate body preferably is preferably sufficiently rigid to permit the catheter to be inserted and withdrawn without the position of the elongate body or the electrode to be moved during insertion or withdrawal of the catheter. The elongate body is preferably sufficiently flexible to permit the catheter to move along with movement of the brain relative to the burr hole so that the elongate body does not damage brain tissue when implanted.

One of skill in the art will understand that the rigidity and flexibility of the elongate body will depend on the material from which the elongate body is formed as well as the thickness of the elongate body. The elongate body may have any suitable thickness from an outer surface to the lumen. In embodiments, the elongate body has a thickness from about 0.1 mm to about 1 mm, such as from about 0.2 mm to about 0.5 mm.

The elongate body may be formed from any suitable material. For example, the elongate body may be formed from polydimethylsiloxane (PDMS).

The opening of the upper flange is preferably aligned with a burr hole in the skull of the subject when the device is implanted. At least a portion of the upper flange extends from the opening such that the upper flange may rest on the skull adjacent to the burr hole. The upper flange may surround the opening. For example, the upper flange may be annular and extend 360 degrees around the opening. The upper flange may extend around the opening less than 360 degrees. For example, the upper flange may extend around the opening from about 10 degrees to about 350 degrees, such as from about 20 degrees to about 340 degrees, from about 30 degrees to about 300 degrees, from about 40 degrees to about 270 degrees, from about 50 degrees to about 240 degrees, or the like. In embodiments, the upper flange extends around the opening from about 90 degrees to about 180 degrees.

At least a portion of the periphery of the upper flange extends from the opening a sufficient distance such that a bottom surface of the portion of the upper flange may rest on the scalp when the opening is aligned with the burr hole. In embodiments, at least a portion of the periphery of the upper flange extends from the opening a distance from about 3 mm to about 15 mm, such as from about 5 mm to about 14 mm, or from about 10 mm to 13 mm.

The upper flange is preferably sufficiently thin to be comfortably tolerated by the subject when implanted. In embodiments, the upper flange is thicker towards the geometric center of the upper flange and tapers to a smaller thickness towards a periphery of the upper flange. Such tapering may be perceived to be more comfortable by the subject or may cause less tissue erosion than an un-tapered profile. The upper flange may comprise a convex top surface.

The bottom surface of the upper flange may be generally flat. For purposes of the present disclosure, “generally flat,” in the context of the bottom surface of the upper flange includes slightly curved to approximate curvature of a skull.

In embodiments, the upper flange may have a maximum thickness of 7 mm or less, such, 6 mm or less, 5 mm or less, 4 mm or less, 3 mm or less, or 2 mm or less. In embodiments, the upper flange may have a maximum thickness of 1 mm or more. In embodiments, the upper flange has a maximum thickness from about 3 mm to about 8 mm. For example, the upper flange may have a maximum thickness in a range from about 4 mm to about 6.5 mm or from about 5 mm to about 6.5 mm.

In embodiments, at least a portion of the periphery of the upper flange has a thickness of 3 mm or less or 2 mm or less. In embodiments, at least a portion of the periphery of the upper flange has a thickness of 1 mm or more. In embodiments, at least a portion of the periphery of the upper flange has a thickness from about 1 mm to about 3 mm.

The upper flange may be formed, at least in part, from a rigid material or materials, such as a hard plastic, ceramic, glass, or metallic material, or a combination thereof. Rigid material may have a higher durometer than more flexible materials. Examples of materials that may be used to form the upper flange or a portion thereof include one or more of a high-performance thermoplastic or relatively rigid plastic material, such as polyurethane, polycarbonate, polysulfone, polyether ether ketone (PEEK), nylon, and Ultra High Molecular Weight Polyethylene (UHMWPE); and a biocompatible metal, such as a stainless steel alloy, titanium, and nitinol. Preferably, the material is compatible with magnetic resonance imaging (MRI). Preferably, the upper flange comprises a biocompatible material or comprises an exterior biocompatible coating.

In embodiments, the upper flange is formed, at least in part, from one or more materials that are softer, more compliant, or softer and more compliant than the rigid material. Examples of suitable soft materials include a thermoplastic elastomer and silicone.

In embodiments, the upper flange comprises a rigid material that is at least partially surrounded by soft material. For example, the soft material may be over-molded over the rigid material. The soft material is preferably biocompatible.

The soft material may serve to absorb impact that may occur to the subject’s skin over the upper flange, which may reduce potential damage to the device or the subject. The soft material may serve to reduce tissue erosion over time, particularly if the soft material is formed from lubricious material or a material that is softer or more compliant than the rigid material. The soft material may comprise one or more antimicrobial agent, such as antimicrobial silver and antibiotics. For example, the soft material may be impregnated with one or antibiotics, such as minocycline and rifampin.

The soft material or the rigid material may be coated with or comprise a lubricious material, such as a hydrogel, an alginate, or the like.

The upper flange may define one or more screw holes through which screws may be used to anchor the upper flange to the skull of the subject. In embodiments, the upper flange does not define a screw hole. If the upper flange does not include a screw hole or other suitable anchoring mechanism, the upper flange may be retained, for example by forceps or another suitable instrument, as the catheter is inserted through, or withdrawn from, the device.

The upper flange may define a recess along at least a portion of the top surface of the upper flange. The recess may extend from the opening towards a periphery of the upper flange. The recess may be configured to loosely receive the catheter. The catheter may exit the opening and be bent into the recess. Preferably, the recess defines a path such that the catheter is curved, as opposed to forming a right-angle bend, when the catheter is pressed against a surface of the upper flange defined by the recess. The recess may have any suitable radius of curvature adjacent the opening.

Preferably, the recess is configured to receive but not retain the catheter. That is, the catheter is preferably freely slidable in the recess. The material defining the recess and the material defining an outer surface of the catheter preferably have a low coefficient of friction to allow the catheter to easily slide along the recess. Preferably, the recess comprises a smooth surface. The surface of the upper flange that defines the recess may be lubricious or a lubricious coating may be applied to the surface of the recess. Examples of lubricious coatings that may be used to coat the lumen include a hydrogel.

The recess preferably has a width greater than the outer diameter of the catheter. In embodiments the recess has a width from about 2 mm to about 5 mm, such as from about 2.2 mm to about 4 mm, or from about 2.5 mm to about 3.5 mm.

The recess may be configured to receive a cable comprising conductors electrically connected to one or more electrodes. The recess may be configured to receive the cable and the catheter.

The device may comprise a mid-portion between the upper flange and the elongate body. The mid-portion comprises a body defining a cavity in communication with the opening of the upper flange and the lumen of the elongate body. An outer surface of the mid-portion may be sized and shaped to be disposed within a burr hole in a skull of a subject. Accordingly, the size and shape of the mid-portion may vary depending on the size and shape of the burr hole, or the size of the burr hole may be determined by the size and shape of the mid-portion. Preferably, the clearance between an exterior side surface of the mid-portion and the skull is small when the mid-portion is positioned in the burr hole. Smaller clearances between the exterior side surface of the mid-portion and the burr hole may improve stability of the device relative to the skull. Improved stability may be manifest in less movement of the device relative to the skull.

In some embodiments, the clearance between the exterior side surface of the mid-portion and the skull, when the mid-portion is disposed in the burr hole, is between 0 mm and 5 mm. For example, the clearance between the exterior side surface of the mid-portion and the skull, when the mid-portion is disposed in the burr hole, may be between 0.1 mm and 3 mm, such as between 0.2 mm and 2 mm.

In some embodiments, the mid-portion has a diameter in a range from about 3 millimeters to about 20 millimeters. For example, the mid-portion of the housing may have a diameter in a range from about 5 millimeters to about 14 millimeters, such as from about 11 millimeters to about 13 millimeters. Typically, the outer surface of the mid-portion is generally cylindrical. The mid-portion of the device may have a generally circular cross-section. In some embodiments, the burr hole may have a diameter of about 14 millimeters. In some embodiments, the burr hole may have a diameter of about 5 millimeters.

When the device is implanted, a bottom major surface of the mid-portion preferably does not extend substantially beyond the bottom of the burr hole. Accordingly, the length of mid-portion of the device may vary depending on the thickness of the skull of the subject into which the device is implanted. As an example, a thickness of a human adult skull may typically be in a range from about 6.5 millimeters to about 7 millimeters.

In some embodiments, the length of the mid-portion of the device is in a range from about 3 millimeters to about 7 millimeters. For example, the length of the mid-portion may be in a range from about 4 millimeters to about 6 millimeters or from about 4.5 millimeters to about 5.5 millimeters.

The cavity of the mid-portion may be configured to receive a portion of a device that is operatively coupled to the catheter. For example, the cavity of the mid-portion may be sized and shaped to receive a portion of a VPS that contains a valve. The cavity of the mid-portion may be sized and shaped the same as or substantially the same as the portion of the device that it is configured to receive.

In some embodiments, the cavity of the mid-portion is configured to receive the catheter. The cavity of the mid-portion may have a diameter that is substantially the same as the lumen of the elongate body. The cavity of the mid-portion may have an inner diameter that is larger than the lumen of the elongate body. In embodiments, the diameter of the cavity of the mid-portion is from about 2 mm to about 13 mm, such as from about 2.5 mm to about 10 mm.

The mid-portion may be made from any suitable materials or material. The mid-portion may be formed, at least in part, from a rigid material or materials, such as a hard plastic, ceramic, glass, or metallic material, or a combination thereof. For example, the mid-portion, at least in part, may comprise one or more of a high-performance thermoplastic or relatively rigid plastic material, such as polyurethane, polycarbonate, polysulfone, polyether ether ketone (PEEK), nylon, and Ultra High Molecular Weight Polyethylene (UHMWPE); and a biocompatible metal, such as a stainless steel alloy, titanium, and nitinol. Preferably, the material is compatible with magnetic resonance imaging (MRI). Preferably, the mid-portion comprises a biocompatible material or comprises an exterior biocompatible coating.

The material or materials of the mid-portion that define the cavity may be lubricious or may comprise a lubricious coating. Examples of lubricious coatings that may be used to coat the lumen include a hydrogel, an alginate, or the like.

The elongate body of the device, regardless of whether the device comprises a mid-portion, is preferably sufficiently long to extend to the surface of the brain or into the brain when the device is implanted. The electrode may be positioned in proximity to a distal end of the elongate body. When the device is implanted, the electrode is preferably on the surface of the brain or is positioned in brain parenchyma.

In embodiments, the elongate body has a length of greater than 5 mm, such as greater than 10 mm, or greater than 15 mm. In embodiments, the elongate body has a length less than 35 mm. In embodiments, the elongate body has a length from about 10 mm to about 20 mm when the device does not have a mid-portion. In embodiments, the elongate body has a length from about 2 mm to about 15 mm when the device comprises a mid-portion.

The device may comprise one or more electrodes disposed on the elongate body. One or more electrodes may be an EEG recording electrode. One or more electrodes may be a ground or reference electrode. In embodiments, a ground or reference electrode may be positioned on another device.

The device may comprise any suitable number of electrodes, such from 1 electrode to about 64 electrodes. For example, 2 to 32 electrodes, such 2 to 16 electrodes, or 2 to 10 electrodes may be disposed on the elongate body.

The electrodes may be longitudinally spaced at any suitable distance or interval. The electrodes may be evenly spaced or unevenly spaced. The spacing between electrodes may vary depending on electrode width, number of electrodes, and desired distance that the electrodes span. The electrodes may span any suitable distance. For example, the electrodes may span a distance of about 5 centimeters, such as about 4 centimeters.

The electrodes may have any suitable width. The electrodes may have the same or different widths. As an example, the electrodes may have a width of from about 0.5 millimeters to about 2 millimeters, such as from about 0.7 millimeters to about 1.5 millimeters.

In embodiments, most or all the electrodes are positioned such that they will be placed in white or grey matter of the brain when implanted. However, it is not necessary that all the electrodes be placed in the white or grey matter. If an electrode is not placed in white or grey matter, the recording from that electrode may continue to be captured and potentially ultimately ignored. Alternatively, recording from that electrode may be inactivated. In embodiments, one or more electrodes are placed on a surface of the brain or above the brain as a ground or reference electrode.

Preferably, a majority of the electrodes are configured to be placed in white or grey matter when the catheter is implanted. Preferably, at least 70% of the electrodes are placed in white or grey matter when the catheter is implanted.

Preferably, at least two electrodes are configured to be placed in white or grey matter when the catheter is implanted. When multiple electrodes record signals from white or grey matter, coherent changes in activity between electrodes may be a powerful way to track more global changes. In some embodiments, the excitable state of a neural network is determined by monitoring a small neuronal population. The more excitable the small neuronal population, the higher the probability for activity to propagate throughout the network causing an ‘avalanche’ of activity. Such monitoring may be valuable for general brain state monitoring and may be particularly valuable for monitoring a brain state to predict a seizure.

The electrodes and associated signal processing apparatus may be configured in any suitable manner. For example, the electrodes and associated signal apparatus may be configured in differential mode or referential mode.

In differential mode, the system comprises an active electrode, a reference electrode, and a ground. The signal difference between an active electrode and a reference electrode may be amplified. The reference electrode may be a common reference for more than one active electrode. The reference electrode is preferably positioned a substantial distance from an active electrode and from the ground. In differential mode, the system may be configured to detect small differences between electrode pairs and may be less likely to be affected by large artifacts originating near the ground electrode. However, the system may not be particularly effective at detecting larger common signals.

Preferably, the system is configured to detect larger common signals. Larger common signals may be associated with an overall brain state or with a seizure.

To detect larger common signals, the system may be configured in referential mode, which may also be referred to as single-ended mode. Referential mode may use a single active electrode per amplifier. There may be more than one active electrode. In referential mode, the output of the active electrode is amplified relative to the ground electrode, as opposed to the reference electrode in differential mode. The ground is preferably placed a substantial distance from the active electrode, which may result in amplification of signals that affect larger parts of the brain. While being effective at detecting larger common signals, referential mode may be sensitive to artifacts. Proper placement of the ground electrode may mitigate some issues associated with artifacts.

In referential mode, the catheter or the brain lead associated with the catheter comprises the active electrode. The catheter or the brain lead associated with the catheter may comprise the ground electrode. The ground electrode may be separate from the catheter or brain lead. Preferably, the ground electrode is placed near the proximal end of the elongate body, such as on the upper flange or mid portion.

The electrodes may be positioned on the elongate body in any suitable manner. For example, the electrodes may extend around the circumference of the elongate body or may extend less than all the way around the circumference of the elongate body. For example, the electrodes may radially extend around the elongate body from about 10 degrees to about 360 degrees, such as from about 10 degrees to about 20 degrees, from about 150 degrees to about 210 degrees, or about 180 degrees. Each electrode may extend around the elongate body the same or a different amount. Preferably, each electrode extends around the elongate body the same amount. Preferably, each electrode extends radially around the elongate body about 180 degrees, or about half-way around the circumference of the elongate body. In some embodiments, every other electrode along the length of the elongate body faces in substantially the same direction. Each subsequent electrode may face a substantially opposite direction as the adjacent electrode.

The electrodes may be made of any suitable material. Suitable materials for implantable electrodes are well-known to those of skill in the art. Materials suitable for deep brain stimulation electrodes are suitable materials for electrodes of the catheters described herein. In embodiments, the electrodes are made from platinum or a platinum iridium alloy.

The electrodes may have any suitable thickness. For example, the electrodes may have a thickness from about 100 microns to about 3 millimeters, such as from about 200 microns to about 2 millimeters. The electrodes may be formed from a foil.

Each electrode may be discretely electrically coupled to one or more electrical contacts. Each electrode may be electrically coupled to a discrete contact by an electrical conductor, such as a wire. The conductors may extend within a body portion of the elongate body. The conductors may branch off from the elongate body in proximity to the proximal end of the elongate body. The conductors may comprise an electrically insulating coating or outer surface. Conductors that branch off the elongate body may be braided or twisted to form a cable. A lead may comprise the cable that branches off the elongate body. The lead may comprise an electrically insulating outer material. The lead may comprise the contacts.

The bottom surface of the upper flange may comprise a groove configured to receive the branched off conductors or lead to allow a proximal end of the conductors or lead to exit the burr hole and be positioned between the scalp and the skull of the subject.

The conductors may extend within the body portion of the elongate body, within a body of a mid-portion, if present, and through a body of the upper flange. The upper flange may comprise an aperture through which the conductors or a lead may exit.

An electrical interconnect may comprise the contacts. The electrical interconnect may allow for electrical connection with another device, such as signal apparatus, or cable for connection to another device. The upper flange may comprise the electrical interconnect.

The signal apparatus may comprise a power source, such as a battery, which may be rechargeable, or may be wirelessly powered. If the signal apparatus is wirelessly powered, the signal apparatus preferably includes an inductive coil, solenoid, or other suitable components to be wirelessly powered by an external apparatus and to transmit data regarding the signals recorded by the electrodes to the external apparatus.

The signal apparatus may be implanted in the subject at any suitable location. In embodiments, the signal apparatus is incorporated into the device. For example, the signal apparatus, or a portion thereof, may be housed in the upper flange of the device.

In embodiments, the signal apparatus is implanted at a location where it may inductively couple with a device external to the subject. For example, the signal apparatus may be positioned under the scalp of the subject near an ear of the subject. Such positioning may allow the external device to be comfortably worn on or around the ear of the subject to provide suitable inductive coupling to power the signal apparatus and to wirelessly transmit data regarding the signals recorded by the electrodes from the signal apparatus to the external device. The external device may then transfer the data directly to the cloud or via another device, such as a smart phone, a personal computer, or the like, which may then transfer the data to a server in the cloud, or the like.

The signal apparatus may be configured to continuously transmit iEEG data derived from an intracranial electrode. The signal apparatus may be configured to continuously transmit the iEEG data for a long duration of time. The signal apparatus may be configured to transmit relatively unfiltered data containing a broad amount of relevant brain signal. That is, the signal apparatus may transmit data regarding a majority of the captured iEEG data. For example, the iEEG data corresponding to the transmitted data may not have been bandpass filtered. As another example, the subsets of the iEEG data are not extracted for transmission. Rather, the majority of the iEEG data is transmitted by the signal apparatus. The signal apparatus may be configured to continuously transmit data for 1 day or more, 1 week or more, 1 month or more, or 1 year or more.

The external device may be configured to continuously receive iEEG data derived from an intracranial electrode. The external device may be configured to continuously receive the iEEG data for a long duration of time. The external device may be configured to continuously receive data for 1 day or more, 1 week or more, 1 month or more, or 1 year or more.

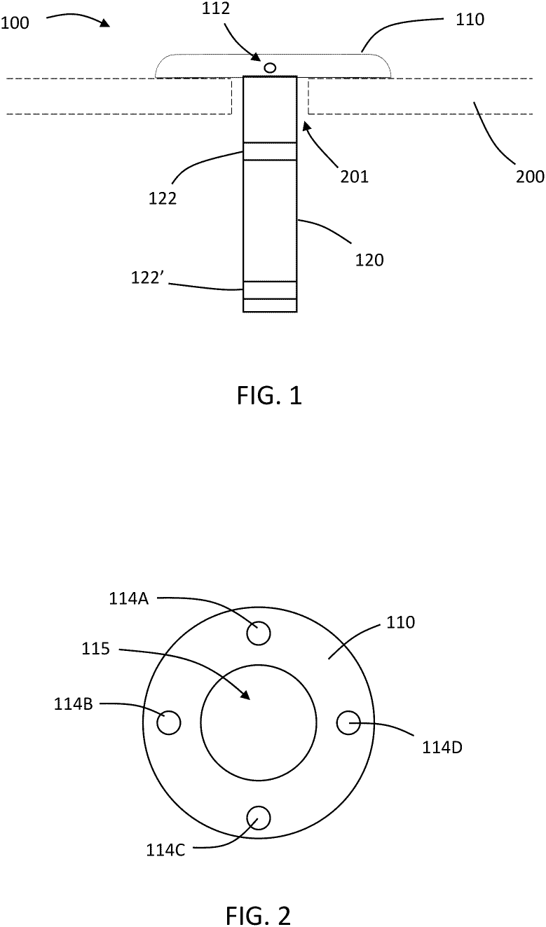

Referring now to FIG. 1, a schematic side view of a device 100 according to an embodiment is shown. The device 100 includes an upper flange 110 and an elongate body 120 coupled to the upper flange 110. First 122 and second 122′ electrodes may be disposed on the elongate body 120. Preferably, at least one of the first 122 and second 122′ electrodes is positioned to contact a brain of the subject when the device 100 is implanted.

The dashed lines in FIG. 1 indicate a skull 200 of the subject, which includes a burr hole 201. A bottom surface of the upper flange 110 rests on the skull 200 adjacent the burr hole 201. The elongate body 120 extends through the burr hole 201.

The upper flange 110 defines an aperture 112 through which a cable or lead (not shown) comprising conductors that are discretely connected to the electrodes 122, 122′ may run such that the cable or lead may be positioned between the skull 200 and a scalp of the subject. Alternatively, the conductors may terminate in an electrical interconnect (not shown), which may be positioned similarly to aperture 112.

The thickness of the upper flange 110 tapers towards the periphery.

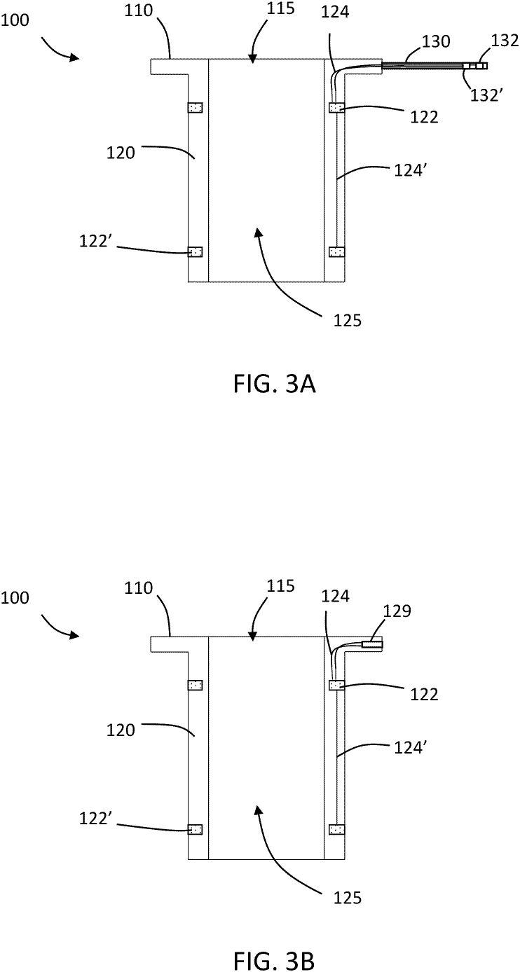

FIG. 2 shows a schematic top view of an embodiment of an upper flange 110. The upper flange 110 comprises screw holes 114A-D for anchoring the upper flange 110 to the skull of the subject. In embodiments (not shown), the upper flange may comprise no screw holes. The upper flange 110 defines an opening 115 through which a catheter may be inserted or withdrawn.

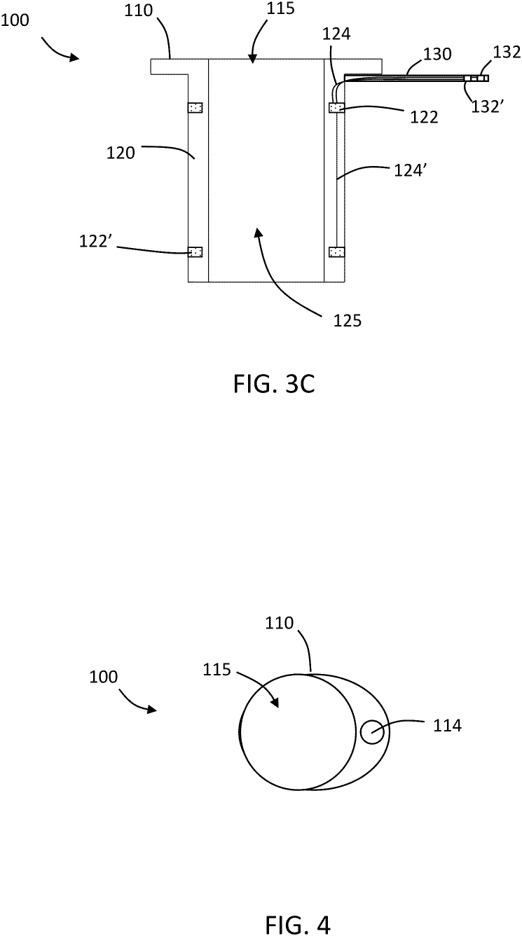

FIGS. 3A-C are schematic sectional views of embodiments of devices 100. The device 100 includes an upper flange 110 and an elongate body 120 extending from the upper flange 110. First 122 and second 122′ electrodes are disposed about the elongate body 120. Conductor 124′ is electrically coupled electrode 122′, and conductor 124 is electrically coupled to electrode 124. In FIG. 3A, a lead 130 exits the upper flange 110 (e.g., through aperture 112 shown in FIG. 1). Conductors 124, 124′ run through the lead. The lead 130 has contacts 132 and 132′ which conductors 124 and 124′, respectively, electrically couple to electrodes 122 and 122′ .

In FIG. 3B, the conductors 124, 124′ terminate at electrical interconnect 129. The electrical interconnect 129 comprises contacts (not shown) to provide connection of a separate component, such as signal apparatus, to electrodes 122, 122′ through a lead operatively coupled to the separate component.

In FIG. 3C, the conductors 124, 124′ run through a portion of a body of the elongate body 120 and branch off from the elongate body 120 in proximity to the proximal end of the elongate body 120 in the form of a lead 130. The lead 130 has contacts 132 and 132′ which conductors 124 and 124′, respectively, electrically couple to electrodes 122 and 122′. The bottom surface of the upper flange 110 may comprise a groove (not shown) configured to receive the lead 130.

In FIGS. 3A-C the upper flange 110 includes an opening 115 in communication with a lumen 125 of the elongate body 120. A catheter may be inserted through opening 115 and lumen 125 to be positioned within the brain of a subject. For example, the catheter may be positioned within a CSF containing space of the subject, such as a cerebral ventricle.

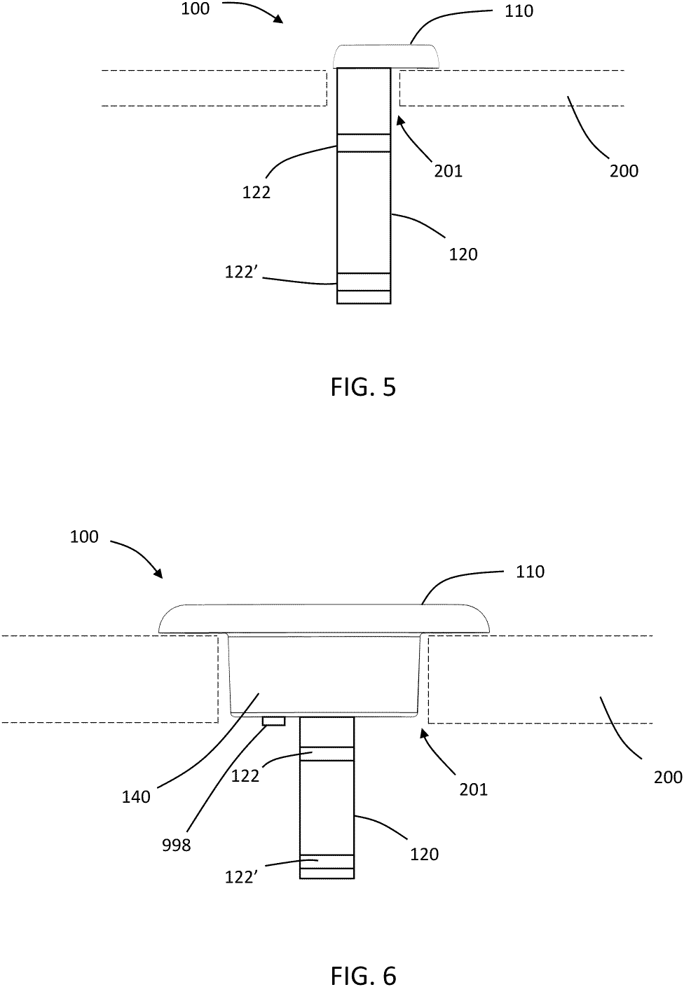

FIG. 4 is a schematic top view of an embodiments of a device 100 showing the upper flange 110. The upper flange 110 extends less than 360 degrees around the opening 115. In the depicted embodiment, the upper flange 110 extends about 90 degrees around the opening 115. The upper flange 114 comprises a screw hole for use with a screw to anchor the upper flange 110 to the skull of the subject.

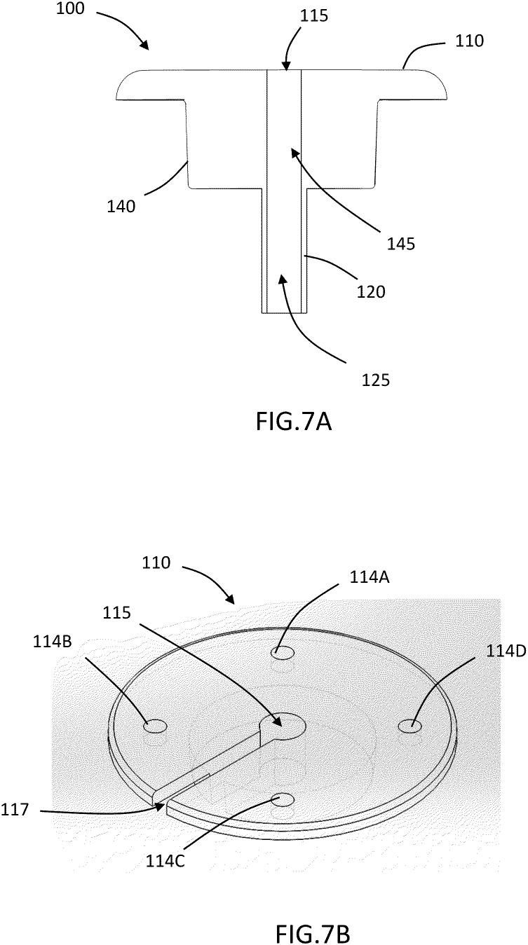

FIG. 5 shows a schematic side view of an embodiment of a device 100 that includes an embodiment of an upper flange 110 shown in FIG. 4. The elongate body 120 extends from the upper flange 110. First 122 and second 122′ electrodes are disposed on the elongate body 120. Preferably, at least one of the first 122 and second 122′ electrodes is positioned to contact a brain of the subject when the device 100 is implanted.

The dashed lines in FIG. 5 indicate a skull 200 of the subject, which includes a burr hole 201. A bottom surface of the upper flange 110 rests on the skull 200 adjacent the burr hole 201. The elongate body 120 extends through the burr hole 201.

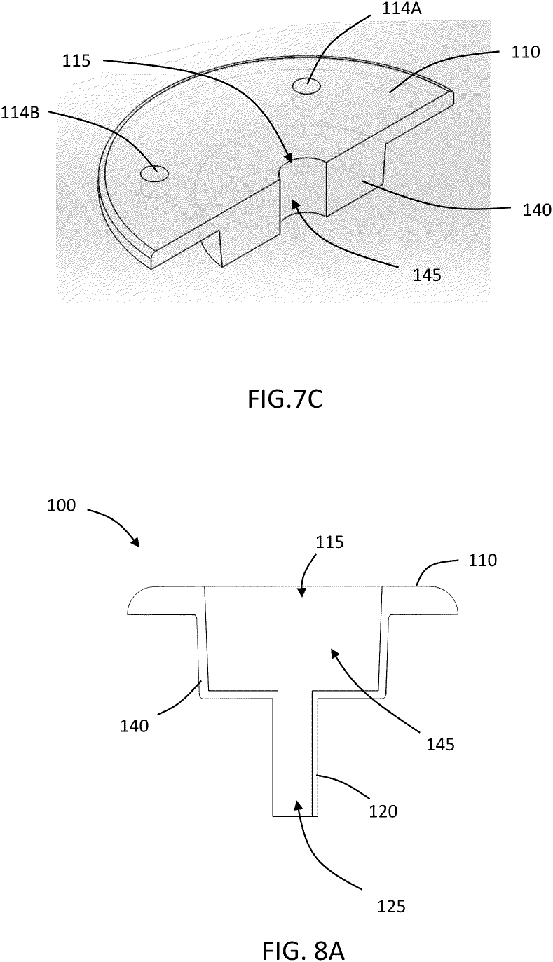

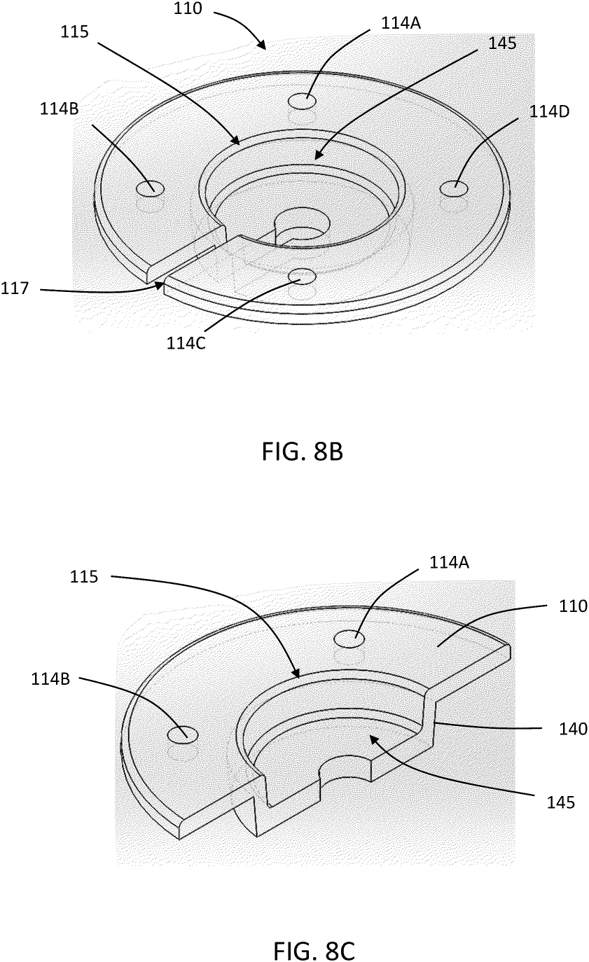

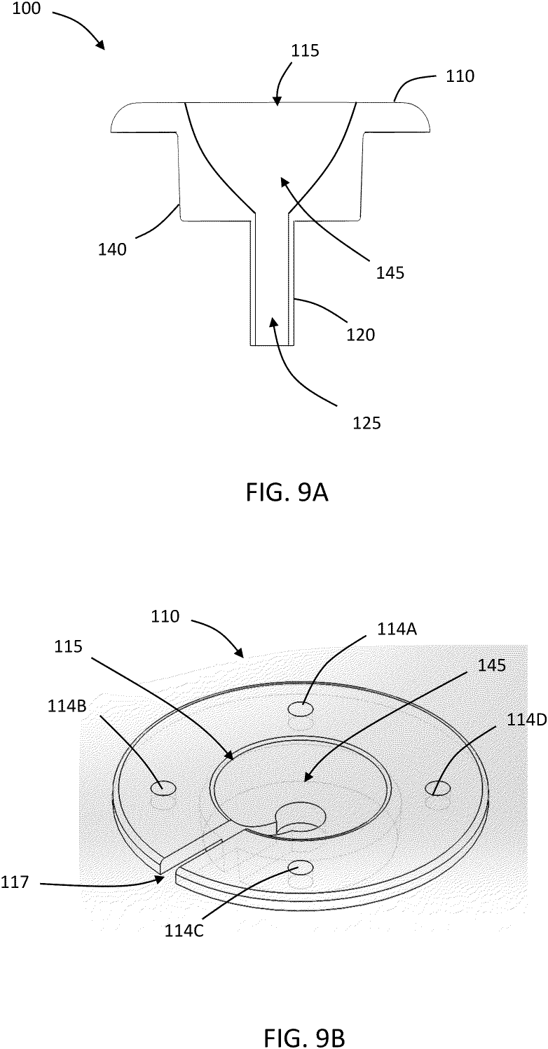

FIGS. 6, 7A-C, 8A-C, and 9A-C show embodiments of devices 100 comprising a mid-portion 140. FIGS. 7A, 8A, and 9A do not show electrodes 122, 122′ for purposes of convenience. FIGS. 7B, 7C, 8B, 8C, 9B, and 9C do not show elongate body 120. The mid-portion 140 is between the upper flange 110 and the elongate body 120. The mid-portion 140 may be positioned in a burr hole 201 in the skull 200 of a subject. The mid-portion has a diameter that is greater than the elongate body 120. The bottom surface of the upper flange 110 rests on the skull 200 adjacent the burr hole 201. The elongate body 120 extends below the skull 200 so that electrodes 122, 122′ may be positioned in or on the brain of the subject.

The mid-portion 140 defines a cavity 145 in communication with the opening 115 of the upper flange 110 and the lumen 125 of the elongate body 120. The cavity 145 may be sized and shaped to receive a portion of a device operatively coupled to a catheter (see, e.g., FIGS. 8 and 9). For example, the cavity may be sized and shaped to receive a portion of a VPS comprising a valve.

The cavity 145 may be substantially the same diameter as the lumen 125 of the elongate body 120 (see, e.g., FIG. 7) such that a catheter may be inserted through the opening 115, the cavity 145, and the lumen 125 into the brain of the subject.

The upper flange 110 may comprise one or more screw holes 114A-D for anchoring the upper flange 110 (and device) to the skull 200. The upper flange 111 may define a recess 117 for receiving a catheter, such as a catheter of a VPS or an EVD. The recess 117 extends from the opening 115. In embodiments, the recess 117 does not securely engage the catheter so that the catheter may freely slide within the recess 115. A width of the recess may be greater than a width of the catheter that it is configured to receive. The material defining the recess 117 may be lubricious, the recess 117 may be coated with a lubricious material, or the recess 117 may be formed from material having a low coefficient of friction with the material from which an exterior surface of the catheter is formed. In embodiments, where the device 110 is configured to receive a catheter of an EVD, the catheter is configured to freely slide within the facilitate explant of the EVD and withdrawal of the catheter. The EVD or catheter may exit the scalp at a substantial distance from the location of the opening 115. For example, the catheter or EVD may exit the scalp at a distance of 10 cm or more or 15 cm or more from the opening 115. Accordingly, allowing the catheter to freely slide within the recess 117 may facilitate withdrawal of the catheter from the device 100 when the EVD or catheter is pulled from an exit location that is at a distance from the device 110.

An electrode 998 may be placed on the mid portion 140, such as on the bottom surface of the mid portion 140 as shown in FIG. 6. The electrode 998 may serve as one or more of a recording electrode, a ground electrode, a return electrode, and a reference electrode.

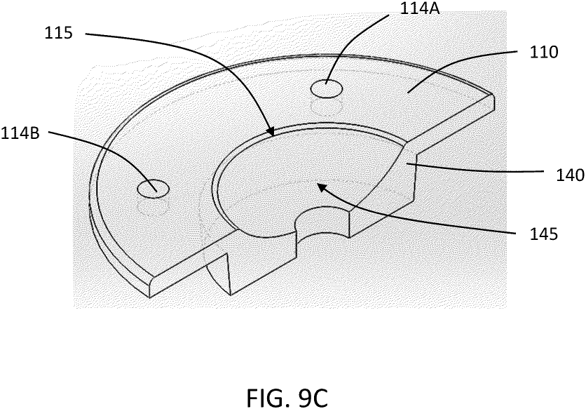

FIG. 10 is a schematic side view of a portion of an embodiment of a VPS 300. The VPS includes a distal catheter 310 having a distal end 312 configured to be implanted in a cerebral ventricle, such as a lateral ventricle and a proximal end configured to be coupled to a valve portion 320. The distal catheter 310 includes a plurality of apertures 316A-D through which CSF may flow. The valve portion 320 comprise a valve that may regulate flow of the CSF. A proximal catheter 330 (only a portion shown) may extend to a peritoneal cavity for draining CSF. The valve portion 320 may be received in a cavity (e.g. cavity 145) of a mid-portion (e.g., mid-portion 140) of a device as shown in, for example, FIG. 9 or other suitably shaped cavity. Accordingly, the distal catheter 310 may be inserted through the lumen (e.g., lumen 125) of the elongate body (e.g., elongate body 120) of, for example the device shown in FIG. 9.

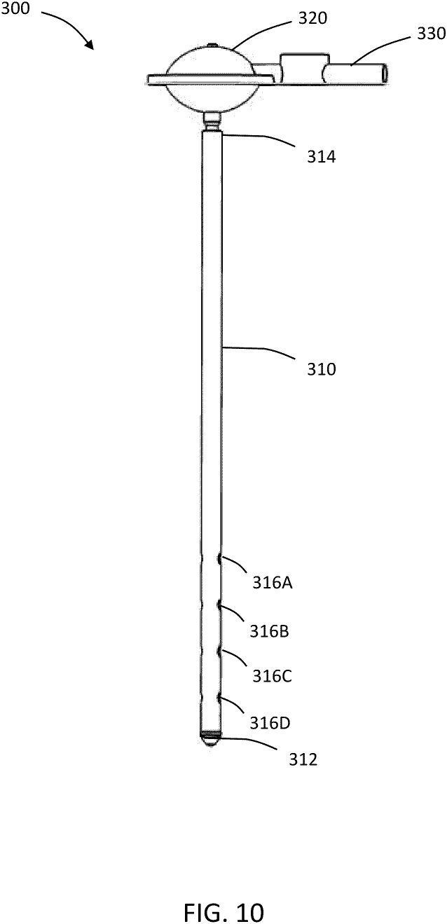

FIG. 11 is a schematic side view of a portion of an embodiment of an VPS 301. The VPS 301 includes a distal catheter portion 310 having a distal end 312 configured to be implanted in a cerebral ventricle, such as a lateral ventricle. The distal catheter portion 310 is connected to a valve portion 320, which may optionally comprise an antechamber. The distal catheter 310 includes a plurality of apertures 316A-D through which CSF may flow. The valve portion 320 comprise a one-way valve to allow CFS to flow through apertures 316A-D through the valve to a proximal end portion 314 of the catheter to shunt CSF within the subject. The distal catheter portion 310 of the VPS 301 may be inserted through a lumen (e.g., lumen 125) of an elongate body (120) of a device as shown in, for example, FIGS. 3 and 7.

While not shown, it will be understood that a external ventricular drain (EVD) may comprise a distal catheter portion similar to the distal catheter portions of the VPS devices shown in FIGS. 10 and 11, which may be inserted through a lumen of an elongate body of a device as described herein. An EVD may comprise a controllable valve external to the subject.

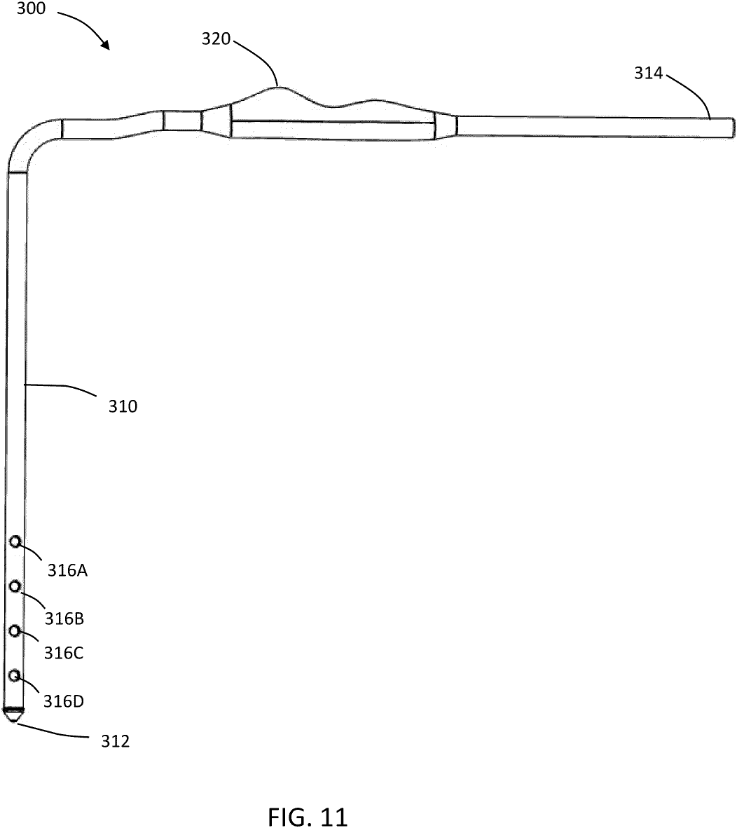

FIG. 12 shows a device 100 comprising an upper flange 111 having a recess 117 for receiving a catheter, such as a catheter of a VPS or an EVD. The recess 117 extends from the opening 115.

FIG. 13 shows a device 100 comprising an upper flange 110 having a bottom surface 118 comprising a groove 119. The groove 119 may be configured to receive a cable or lead comprising conductors electrical coupled to electrodes 122, 122′ that are disposed on the elongate body 120, which defines lumen 125. The groove 119 may be configured to receive a cable or lead (e.g., lead 130 in FIG. 3) that branches off from the elongate member 120 (cable or lead not shown in FIG. 13 to more clearly show groove 119).

An electrode 999 may be positioned on the upper flange 110, such as on the bottom surface 118 of the upper flange 110. The electrode 999 may serve as one or more of a reference electrode, a ground electrode, a return electrode, and a recording electrode.

While the upper flange 110 in FIGS. 12 and 13 does not include a screw hole, it will be understood that that the upper flange 110 may include one or more screw holes.

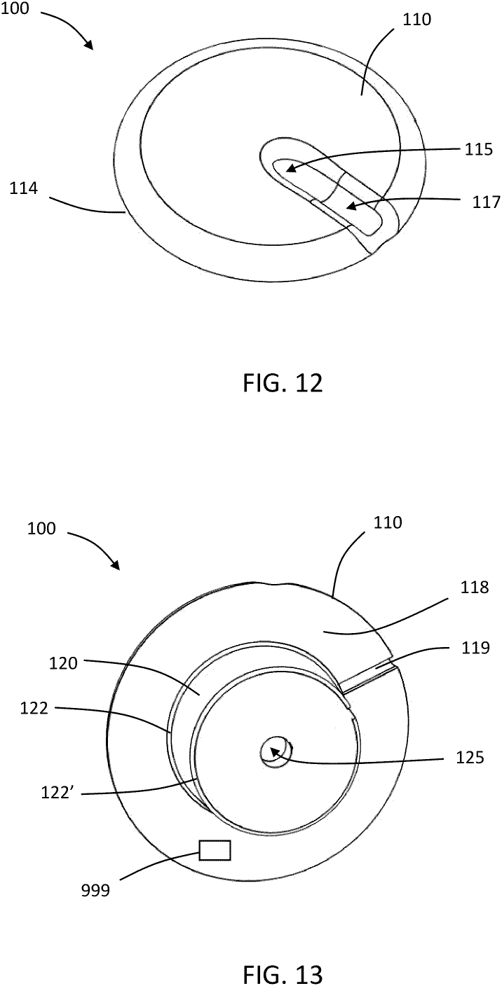

FIG. 14 shows a device 100 implanted in a subject. The upper flange 110 of the device 100 rests on the skull 200, and the elongate body 120 extends through a burr hole in the skull 200 into the brain of the subject. Electrodes 122 and 122′, which are disposed on the elongate body 120, are positioned intracranially, specifically in the brain parenchyma. A lead 130 couples signal apparatus 400 to the electrodes 122 and 122′. The lead 130 may be a lead as shown in, for example, FIG. 3 and may be received in a groove on the bottom surface of the upper flange 110 as shown in, for example, FIG. 13. The lead 130 may exit an aperture (e.g., aperture 112 as shown in, for example, FIG. 1) in the upper flange 110. The lead 130 may be coupled to another lead or to an electrical interconnect in the upper flange 110 to couple the signal apparatus 400 to the electrodes 122 and 122′.

A distal end 312 of the catheter 310 is inserted through the opening (not shown in FIG. 14) in the upper flange 110 and through the lumen (not shown in FIG. 14) and into a lateral ventricle of the brain. The catheter 310 may be a catheter of a VPS (e.g., as shown in FIG. 10) or an EVD (e.g., as shown in FIG. 11). The catheter 310 (or another catheter coupled to the catheter 310 through, e.g., a valve portion) may exit the opening in the upper flange 110 and be bend into a recess (e.g., recess 117 as shown in FIG. 12) and may extend between the scalp 500 and the skull 200 until the catheter exits the skin of the subject at a distance from the device 100, if, for example, the catheter 310 is part of an EVD. CSF may drain from the proximal end portion 314 of the catheter. If the catheter 310 were part of, for example, a VPS, the proximal end of the catheter would not exit the skin of the subject but rather would be tunneled to the peritoneal cavity of the subject where CSF fluid may drain.

An electrode 999 may be positioned on the upper flange 110, such as on a bottom surface of the upper flange 110. The electrode 999 may serve as one or more of a reference electrode, a ground electrode, a return electrode, and a recording electrode.

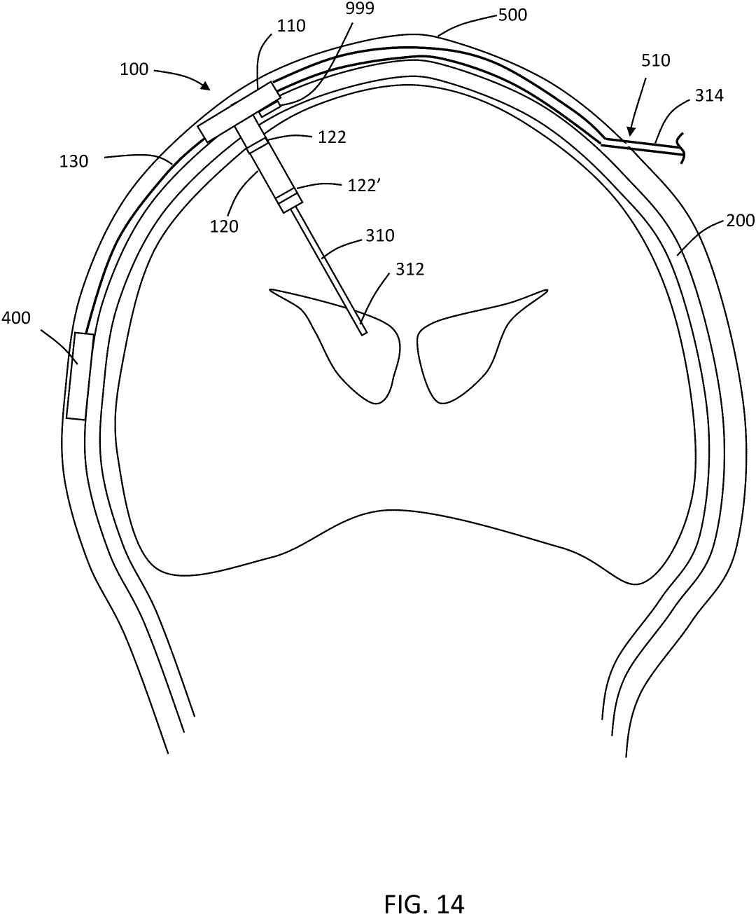

FIG. 15 shows a device 100 relative to a skull 200 of a subject. The upper flange 110 of the device 100 rests on the skull 200, the elongate body 120 extends through a burr hole in the skull 200 into the brain of the subject, and a catheter 310 extends through the device 100. Electrodes 122 and 122′, which are disposed on the elongate body 120, are positioned intracranially. A lead 130 couples signal apparatus 400 to the electrodes 122 and 122′. The lead 130 may be a lead as shown in, for example, FIG. 3 and may be received in a groove on the bottom surface of the upper flange 110 as shown in, for example, FIG. 13. The lead 130 may exit an aperture (e.g., aperture 112 as shown in, for example, FIG. 1) in the upper flange 110. The lead 130 may be coupled to another lead or to an electrical interconnect in the upper flange 110 to couple the signal apparatus 400 to the electrodes 122 and 122′.

The signal apparatus 400 may comprise an electrode 422, which may be positioned intracranially. The electrode 422 may be a recording electrode. The electrode 422 may be a ground or reference electrode relative to, for example, electrode 122 or 122′. The signal apparatus 410 may comprise an upper flange portion 410 and an elongate body portion 420 extending from the upper flange portion 410. The upper flange portion 410 rests on the skull 200, and the elongate body portion 420 extends through a burr hole in the skull 200 such that the electrode 422, which is disposed on the elongate body portion 420 is position intracranially.

The signal apparatus 400 depicted in FIGS. 14 or 15 may transmit data recorded from electrodes 122, 122′, 422, or a combination of two or more thereof to a device external to the subject.

An electrode 999 may be positioned on the upper flange 110, such as on a bottom surface of the upper flange 110. The electrode 999 may serve as one or more of a reference electrode, a ground electrode, a return electrode, and a recording electrode.

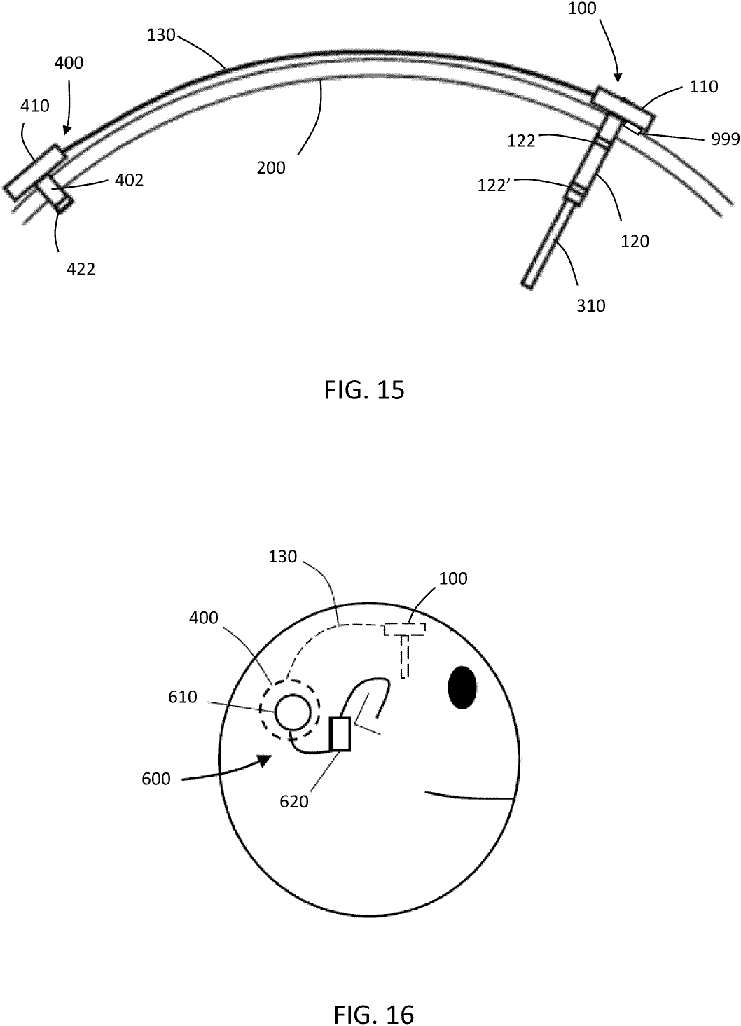

FIG. 16 shows a wearable external device 600 configured to receive data regarding the iEEG signals transmitted from the signal apparatus 400. Implanted components are shown in dashed lines. The implanted system comprises a device 100 as described herein that includes a passageway through which a catheter may be inserted. The device 100 comprises one or more electrodes (not shown) that are implanted intracranially. iEEG signals from the electrodes are transmitted to signal apparatus 400 via a lead 130. The signal apparatus 400 transmits data regarding the iEEG signals to the external device 600.

The external device 600 may comprise an inductive coupling component 610 that may be positioned over the signal apparatus 400 and may comprise a processing component 620 operably coupled to the inductive coupling component 510. The processing component 620 may include, among other things, a rechargeable battery and a processor. The external device 600 may transmit data received from signal apparatus 400, or a processed version thereof, to suitable secondary device. The secondary device or external apparatus 600 may transmit data to the internet, where the data may be stored or retrieved by other computing devices as appropriate. In some embodiments (not shown), the signal apparatus may transmit data directly to the secondary device.

The iEEG data may be used for any suitable purpose. The iEEG data may be used to treat, monitor, or treat and monitor a disease associated with elevated ICP or hydrocephalus, or any other condition for which an externalized ventricular drain (EVD) or ventriculoperitoneal (VP) shunt may be used. The iEEG data may be used to identify, classify, or predict a brain state associated with the disease being treated or that has been treated. The iEEG data may be used to identify, classify, or predict a brain state that is not associated with the disease being treated or that has been treated. The iEEG data may be used to identify, classify, or predict a brain state associated with the disease being treated or that has been treated and a brain state that is not associated with the disease being treated or that has been treated. The iEEG data may be used to identify, classify, or predict a psychological brain state associated with the disease being treated or that has been treated and a brain state that is not associated with the disease being treated or that has been treated.

The iEEG data may be used to develop or train an AI model that may identify, classify, or predict a brain state. The iEEG data may be input into an AI model that may identify, classify, or predict a brain state. The brain state may or may not be associated with the disease being treated or that has been treated. The brain state may be a psychological brain state.

Development, training, refining, and utilizing AI models based on iEEG data to identify, classify, or predict a brain state, such as a psychological brain state are discussed in U.S. Pat. Application No. 17/380,694, entitled MONITORING BASED ON CONTINUOUS INTRACRANIAL EEG ACTIVITY, filed on Jul. 20, 2021, and naming Cerebral Therapeutics, Inc. as an Applicant, and U.S. Provisional Pat. Application No. 63/280,367, entitled DEVELOPMENT AND IMPLEMENTATION OF PSYCHOLOGICAL STATE MODEL, filed on Nov. 17, 2021, and naming Cerebral Therapeutics, Inc. as an Applicant, which provisional applications are hereby incorporated herein by reference in their entireties to the extent that they do not conflict with the disclosure presented herein.

The devices and systems described herein may be implanted in any suitable subject. The subject may experience elevated intracranial pressure (ICP) for which implantation of an externalized ventricular drain (EVD) or ventriculoperitoneal shunt (VPS) is indicated. To date, while EVDs and VPSs are surgically implanted in thousands of patients, the devices have been employed and designed solely to lessen ICP. Current EVD and VP devices provide no information about brain physiology and function despite the great need for understanding of evolving brain states or predicting brain states and despite the fact that EVD devices are already being physically implanted in patients’ brains.

Examples of diseases which may be treated by ventriculostomy include those discussed in B.P. Rosenbaum et al., Journal of Clinical Neuroscience 21 (2014) 623-632 (https://doi.org/10.1016/J.JOCN.2013.09.001), such as subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft). Other diseases that may be treated by ventriculostomy include traumatic brain injury (TBI), congenital hydrocephalus, aneurysms, and the like. Diseases treatable by ventriculostomy, e.g., with an EVD, may also be treated with a CSF drainage shunt, such as a ventriculoperitoneal (VP) shunt.

In some embodiments, a patient suffering from a disease associated with elevated ICP or hydrocephalus may be treated with an EVD and a VP shunt. The treatment with the EVD and VP shunt may be concurrent or may occur at different times. For example, the patient may first be treated with an EVD and then be treated with a VP shunt.

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

Example 1: A device comprising: an upper flange comprising an opening configured to receive a catheter; an elongate body defining a lumen configured to receive the catheter, wherein the lumen is in communication with the opening of the upper flange; and an electrode disposed on the elongate body, wherein, when the device is implanted in a subject, the upper flange is configured to rest on a skull of the subject adjacent to a burr hole, the elongate body is configured to be inserted through the burr hole, and the electrode is configured to be positioned intracranially in the subject, wherein the electrode is configured to detect intracranial electroencephalogram (iEEG) signals from the subject.

Example 2: The device of Example 1, wherein the catheter is part of a cerebrospinal fluid (CSF) shunt or an external CSF drainage catheter.

Example 3: The device of Example 1, wherein the catheter is part of a ventriculoperitoneal shunt or an external ventricular drainage catheter.

Example 4: The device of any one of Examples 1 to 3, comprising a lubricious coating on a surface of the elongate body defining the lumen.

Example 5: The device of any one of Examples 1 to 4, comprising a conductor operatively coupled to the electrode.

Example 6: The device of Example 5, wherein the conductor extends through at least a portion of the elongate body.

Example 7: The device of Example 5 or 6, wherein the conductor extends to or beyond the upper flange.

Example 8: The device of Example 7, wherein the upper flange comprises a bottom surface having groove, wherein the groove is configured to receive the conductor.

Example 9: The device of Example 7, wherein the upper flange comprises an aperture through which the conductor extends.

Example 10. The device of Example 7, comprising an electrical connector positioned at the upper flange, wherein the electrical connector is operatively coupled to the conductor.

Example 11: The device of any one of Examples 1 to 10, wherein the upper flange surrounds the opening.

Example 12: The device of any one or Examples 1 to 10, wherein the upper flange extends around the opening less than 360 degrees.

Example 13: The device of Example 12, wherein the upper flange extends around the opening from about 90 degrees to about 180 degrees.

Example 14: The device of any one of Examples 1-13, wherein the upper flange comprises a hole configured to receive a screw for securing the upper flange to the skull.

Example 15: The device of any one of Examples 1-14, wherein the upper flange comprises an upper surface defining a recess configured to receive a portion of the catheter, wherein the recess extends from the opening towards a peripheral edge of the upper flange.

Example 16: The device of Example 15, wherein the recess has a width larger than an outer diameter of the catheter such that the recess is configured to loosely receive the portion of the catheter.

Example 17: The device of any one of Examples 1 to 16, further comprising a mid-portion between the upper flange and the elongate body, the mid portion defining a cavity in communication with the opening of the upper flange and the lumen of the elongate body.

Example 18: The device of Example 17, wherein, when the device is implanted, the mid-portion is configured to be received in the burr hole.

Example 19: The device of Example 18, wherein the mid-portion is configured to not extend beyond the burr hole when the device is implanted.

Example 20: The device of any one of Examples 17 to 19, wherein the mid-portion is configured to receive a portion of a device operatively coupled to the catheter.

Example 21: The device of any one of Examples 17 to 19, wherein the mid-portion is configured to receive a portion of a shunt device operatively coupled to the catheter.

Example 22: The device of any one of Examples 17 to 21, comprising a mid-portion electrode, wherein the mid portion electrode is positioned on the mid-portion.

Example 23: The device of Example 22, wherein the mid portion electrode is positioned on a bottom surface of the mid-portion.

Example 24: The device of any one of Examples 1 to 23, wherein the elongate body has a length from about 10 mm to about 20 mm

Example 25: The device of any one of Examples 1 to 24, wherein the elongate body has a thickness defined by a distance from an interior surface defining the lumen to an exterior surface, wherein the thickness is from about 0.2 mm to about 0.5 mm

Example 26: The device of any one of Examples 1 to 25, comprising an upper flange electrode, wherein the upper flange electrode is positioned on the upper flange.

Example 27: The device of Example 26, wherein the upper flange electrode is positioned on a bottom surface of the upper flange.

Example 28: A system comprising: the device of any one of Examples 1 to 27; and a signal apparatus operatively couplable to the electrode of the device.

Example 29: The system of Example 28, wherein the signal apparatus is implantable.

Example 30: The system of Example 29, wherein the signal apparatus is configured to be implanted between a scalp and the skull of the subject.

Example 31: The system of Example 30, wherein the signal apparatus is configured to transmit data regarding the iEEG signals detected by the electrode.

Example 32: The system of any one of Examples 28 to 31, comprising an external device configured to receive data transmitted by the signal apparatus.

Example 33: The system of Example 32, wherein the external device is wearable by the subject.

Example 34: The system of any one of Examples 28 to 33, further comprising the catheter.

Example 35: The system of Example 34, wherein the catheter comprises a distal end configured to be placed in a cerebral ventricle of the subject.

Example 36: The system of Example 34 or 35, comprising a non-catheter component of a CSF shunt.

Example 37: A method comprising: implanting the device of any one of Examples 1-27 in the subject such that the upper flange is rests on the skull of the subject adjacent to the burr hole, the elongate body is inserted through the burr hole, and the electrode is positioned intracranially in the subject.

Example 38: The method of Example 37, comprising anchoring the upper flange to the skull.

Example 39: The method of claim 37 or 38, comprising inserting the catheter through the opening of the flange and the lumen of the elongate body.

Example 40: The method of claim 39, comprising implanting the catheter such that a distal end of the catheter is positioned in a cerebral ventricle of the subject.

Example 41: The method of any one of Examples 37 to 40, comprising explanting the catheter and leaving the device implanted, wherein explanting the catheter comprises withdrawing the catheter through the lumen of the elongate body and the opening of the flange.

Example 42: The method of any one of Examples 37 to 41, comprising transmitting data regarding the iEEG signals to a device external to the subject.

While the devices described herein are in relation to catheters associated with EVDs and VPSs, it will be understood that the devices may be employed with other suitable catheters, such as implantable catheters associated with implantable drug pumps or infusion devices.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. A device comprising:
an upper flange comprising an opening configured to receive a catheter;
an elongate body defining a lumen configured to receive the catheter, wherein the lumen is in communication with the opening of the upper flange; and
an electrode disposed on the elongate body,
wherein, when the device is implanted in a subject, the upper flange is configured to rest on a skull of the subject adjacent to a burr hole, the elongate body is configured to be inserted through the burr hole, and the electrode is configured to be positioned intracranially in the subject, wherein the electrode is configured to detect intracranial electroencephalogram (iEEG) signals from the subject.

2. The device of claim 1, wherein the catheter is part of a cerebrospinal fluid (CSF) shunt or an external CSF drainage catheter.

3. The device of claim 1, comprising a conductor operatively coupled to the electrode.

4. The device of claim 3, wherein the conductor extends through at least a portion of the elongate body.

5. The device of claim 3, wherein the conductor extends to or beyond the upper flange.

6. The device of claim 1, wherein the upper flange surrounds the opening.

7. The device of claim 1, wherein the upper flange extends around the opening less than 360 degrees.

8. The device of claim 1, wherein the upper flange comprises an upper surface defining a recess configured to receive a portion of the catheter, wherein the recess extends from the opening towards a peripheral edge of the upper flange.

9. The device of any claim 1, further comprising a mid-portion between the upper flange and the elongate body, the mid portion defining a cavity in communication with the opening of the upper flange and the lumen of the elongate body.

10. The device of claim 9, wherein, when the device is implanted, the mid-portion is configured to be received in the burr hole.

11. The device of claim 9, wherein the mid-portion is configured to receive a portion of a device operatively coupled to the catheter.

12. The device of claim 9, comprising a mid-portion electrode, wherein the mid portion electrode is positioned on the mid-portion.

13. A system comprising:
the device of claim 1; and
a signal apparatus operatively couplable to the electrode of the device.

14. The system of claim 13, wherein the signal apparatus is implantable.

15. The system of claim 14, wherein the signal apparatus is configured to transmit data regarding the iEEG signals detected by the electrode.

16. The system of claim 13, comprising an external device configured to receive data transmitted by the signal apparatus.

17. A method comprising:
implanting the device of claim 1 in the subject such that the upper flange is rests on the skull of the subject adjacent to the burr hole, the elongate body is inserted through the burr hole, and the electrode is positioned intracranially in the subject.

18. The method of claim 17, comprising inserting the catheter through the opening of the flange and the lumen of the elongate body.

19. The method of claim 18, comprising implanting the catheter such that a distal end of the catheter is positioned in a cerebral ventricle of the subject.

20. The method of claim 17, comprising transmitting data regarding the iEEG signals to a device external to the subject.
